(19) United States
(12) Patent Application Publication
(10) Pub. No.: US 2021/0338639 A1
(43) Pub. Date: Nov. 4, 2021

(54) METHODS OF TREATING COGNITIVE IMPAIRMENT OR IMPROVING COGNITIVE FUNCTION

(71) Applicant: vTv Therapeutics LLC, High Point, NC (US)

(72) Inventor: Maria Carmen Valcarce Lopez, Oak Ridge, NC (US)

(21) Appl. No.: 17/236,235

(22) Filed: Apr. 21, 2021

Related U.S. Application Data
(63) Continuation of application No. PCT/US2019/056854, filed on Oct. 18, 2019.
(60) Provisional application No. 62/748,850, filed on Oct. 22, 2018.
(60) Provisional application No. 62/749,315, filed on Oct. 23, 2018.
(60) Provisional application No. 62/828,080, filed on Apr. 2, 2019.
(60) Provisional application No. 62/898,283, filed on Sep. 10, 2019.

(51) Int. Cl. A61K 31/4174; A61P 25/28

(52) U.S. Cl. CPC A61K 31/4174; A61P 25/28

(57) ABSTRACT

The present invention relates to methods of treating cognitive impairment or improving cognitive function in an individual having poor glycemic control, or having elevated level or one or more RAGE ligands, or having elevated level of one or more inflammatory biomarkers, or overexpressing RAGE, where the method comprises administering [3-(4-{2-butyl-1-[4-(4-chloro-phenoxy)-phenyl]-1H-imidazol-4-yl}-phenoxy)-propyl]-diethyl amine (“COMPOUND I”) or a pharmaceutically acceptable salt thereof.

### FIELD OF THE INVENTION

The present invention relates to methods of treating cognitive impairment or improving cognitive function in an individual by administering [3-(4-{2-butyl-1-[4-(4-chloro-phenoxy)-phenyl]-1H-imidazol-4-yl}-phenoxy)-propyl]-diethyl amine (“COMPOUND I”) or a pharmaceutically acceptable salt thereof.

### BACKGROUND OF THE INVENTION

The Receptor for Advanced Glycation Endproducts (RAGE) is a member of the immunoglobulin super family of cell surface molecules. Activation of RAGE in different tissues and organs leads to a number of pathophysiological consequences. RAGE has been implicated in a variety of conditions including: acute and chronic inflammation, the development of diabetic late complications such as increased vascular permeability, nephropathy, atherosclerosis, and retinopathy. RAGE has also been implicated in Alzheimer's disease.

Binding of ligands such as advanced glycation endproducts (AGEs), S100/calgranulin/EN-RAGE, β-amyloid, CML (N^{ε}-Carboxymethyl lysine), and amphoterin to RAGE has been shown to modify expression of a variety of genes. For example, in many cell types interaction between RAGE and its ligands generates oxidative stress, which results in activation of the free radical sensitive transcription factor NF-κB and the activation of NF-κB regulated genes, such as the cytokines IL-1β, TNF-α, and the like.

The association between diabetes and dementia has been studied, and numerous studies have suggested a link between type 2 diabetes (T2D) and Alzheimer's disease (AD). Recently, a linear correlation between circulating hemoglobin A1c (HbA1c) levels and cognitive decline has been demonstrated in the English Longitudinal Study of Ageing.

The multiligand nature of RAGE is highlighted by its ability to bind diverse ligands such as advanced glycation end-products (AGEs) (linked to diabetic complications) and @-amyloid fibrils (a hallmark of AD). The pathogenic role of RAGE in chronic inflammation is undisputed. RAGE is sharply upregulated in numerous cell types under pathological conditions and is also upregulated by RAGE ligands. The feedback loop of RAGE upregulation between each of these mechanisms perpetuates a cascade of inflammation.

Neuroinflammation has emerged as a key component of AD pathology, and participation of RAGE signaling in neurodegenerative diseases via direct effects on neurons and indirect effects through microglia and astrocyte activation is well documented. Moreover, increased expression of RAGE has been shown postmortem in the brain of AD patients. Similarly, a direct correlation between AGEs and the development and progression of diabetic vascular disease and complications has been reported. The presence of low-level inflammation in diabetic complications is supported by numerous experimental evidence in the macro- and microvasculature.

The role of inflammation and RAGE expression/signaling associated with AD and T2D raises the questions of whether RAGE could be a common denominator between various types of cognitive impairment and poor glycemic control and whether treatment with COMPOUND I, a RAGE-inhibitor, could have a distinct effect in people suffering from both poor glycemic control and cognitive impairment.

Thus, there is a continual needed for developing new and effective methods to treat cognitive impairment or methods to improve cognition or cognitive function in subjects with poor glycemic control. Methods described herein are directed towards these and other ends.

### SUMMARY OF THE INVENTION

The present invention provides a method of treating cognitive impairment or improving cognition in a subject having poor glycemic control comprising administering to the subject an amount of less than 20 mg per day of COMPOUND I or a pharmaceutically acceptable salt thereof.

The present invention also provides a method of treating cognitive impairment or improving cognition in a subject having an elevated level of one or more RAGE ligands comprising administering to the subject an amount of less than 20 mg per day of COMPOUND I or a pharmaceutically acceptable salt thereof.

The present invention also provides a method of treating cognitive impairment or improving cognition in a subject having an elevated level of one or more inflammatory biomarkers comprising administering to the subject an amount of less than 20 mg per day of COMPOUND I or a pharmaceutically acceptable salt thereof.

The present invention also provides a method of treating cognitive impairment or improving cognition in a subject comprising administering to the subject an amount of less than 20 mg per day of COMPOUND I or a pharmaceutically acceptable salt thereof, wherein the level of one or more inflammatory biomarkers is reduced.

The present invention also provides a method of treating cognitive impairment or improving cognition in a subject overexpressing RAGE comprising administering to the subject an amount of less than 20 mg per day of COMPOUND I or a pharmaceutically acceptable salt thereof.

The present invention also provides a method of treating cognitive impairment or improving cognition in a subject comprising selecting a subject for treatment having a diagnosis of having poor glycemic control, of having an elevated level of one or more RAGE ligands, of having an elevated level of one or more inflammatory biomarkers, of having an HbAc1 of greater than or equal to 6.5% and/or of overexpressing RAGE; and administering to the subject an amount of less than 20 mg per day of COMPOUND I or a pharmaceutically acceptable salt thereof.

The present invention also provides a method of treating a subject having cognitive impairment comprising determining whether the subject has poor glycemic control, has an elevated level of one or more RAGE ligands, has an elevated level of one or more inflammatory biomarkers, has an HbAc1 of greater than or equal to 6.5%, and/or is overexpressing RAGE; and if the subject has poor glycemic control, has an elevated level of one or more RAGE ligands, has an elevated level of one or more inflammatory biomarkers, has an HbAc1 of greater than or equal to 6.5% and/or is overexpressing RAGE, then administering to the subject an amount of less than 20 mg per day of COMPOUND I or a pharmaceutically acceptable salt thereof.

In each of the above methods, the subject may exhibit improved cognition or exhibit a reduced rate of cognitive decline while exhibiting no significant or no pronounced improvement in glycemic control.

### BRIEF DESCRIPTION OF DRAWINGS

FIG. 1—An 18-month graph displaying the change from baseline in ADAS-cog11 for subgroup of patients in the A Study having HbA1c of 6.5% or more at baseline. Active N=18; Placebo N=8.

FIG. 2—An 18-month graph displaying the change from baseline in CDR_{sb} for subgroup of patients in the A Study having HbA1c of 6.5% or more at baseline. Active N=18; Placebo N=8.

FIG. 3A—A 12-month graph displaying the change from baseline in ADAS-cog11 for subgroup of patients in the A and B Studies having HbA1c of 6.5% or more at baseline. Active N=33; Placebo N=22.

FIG. 3B-A 12-month graph displaying the change from baseline in CDR_{sb} for subgroup of patients in the A and B Studies having HbA1c of 6.5% or more at baseline.

FIG. 4—An 18-month graph displaying the change from baseline in ADAS-cog11 for subgroup of patients in the A Study having HbA1c of 6.5% or more at any time during study and on stable diabetes therapy. Subjects using insulin were excluded from the subgroup. Active N=26; Placebo N=21.

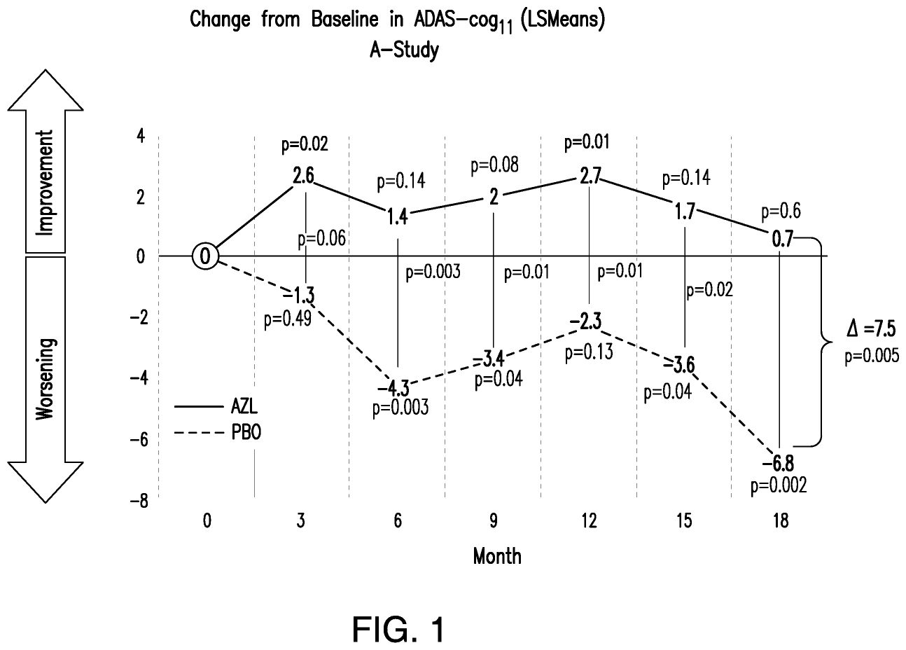

All p-values presented in FIGS. 1-4 are nominal, since the primary analysis of this study was negative.

### DETAILED DESCRIPTION

The present invention relates to treating subjects with cognitive impairment and poor glycemic control with COMPOUND I. The present invention also relates to improving cognition or cognitive function in subjects with poor glycemic control. The present invention also relates to treating cognitive impairment, improving cognition, or improving cognitive function in subjects having elevated levels of one or more RAGE ligands. The present invention also relates to treating cognitive impairment, improving cognition, or improving cognitive function in subjects where the treatment results in reduction in the level of one or more inflammatory biomarkers. The present invention also relates to treating cognitive impairment, improving cognition, or improving cognitive function in subjects overexpressing RAGE. The present invention also relates to methods of selecting a subject for treating cognitive impairment, improving cognition, or improving cognitive function.

The present invention is based on results from two randomized, double-blind, placebo-controlled studies in approximately 800 participants with probable mild AD (MMSE 21-26, CDR global 0.5-1), receiving stable standard of care therapy (acetylcholinesterase inhibitor and/or memantine; SoC) evaluating the efficacy and safety of 18 months of treatment with COMPOUND I (azeliragon) 5 mg/day relative to placebo. The clinical trial design included two separate studies (A-Study and B-Study) operationally conducted under a single protocol. Each study was independently powered to evaluate efficacy with respect to co-primary endpoints of ADAS-cog and CDR-sb and each study was randomized independently. In each study, participants were randomized 1:1 (site-based randomization) to azeliragon (5 mg/day) plus SoC or placebo plus SoC. Primary efficacy outcomes included co-primary endpoints of change from baseline in the ADAS-cog11 at Month 18 and change from baseline in the CDR-sb at Month 18. Secondary endpoints included ADCS-ADL, NPI, MMSE, COWAT, CFT, Trails A and B, RUD-lite, DEMQOL, FDG-PET sub-study, Biomarkers: Plasma A131.40, A131.42.

Methods of Treatment

In a first aspect, the present invention provides a method of treating cognitive impairment in a subject having poor glycemic control comprising: administering to the subject an amount of less than 20 mg per day of COMPOUND I or a pharmaceutically acceptable salt thereof.

In a second aspect, the present invention provides a method of improving cognition or cognitive function in a subject having poor glycemic control comprising: administering to the subject an amount of less than 20 mg per day of COMPOUND I or a pharmaceutically acceptable salt thereof.

In a third aspect, the present invention provides a method of treating cognitive impairment in a subject having an elevated level of at least one RAGE ligand comprising: administering to the subject an amount of less than 20 mg per day of COMPOUND I or a pharmaceutically acceptable salt thereof. In a further embodiment, the subject may have poor glycemic control.

In a fourth aspect, the present invention provides a method of improving cognition or cognitive function in a subject having an elevated level of at least one RAGE ligand comprising: administering to the subject an amount of less than 20 mg per day of COMPOUND I or a pharmaceutically acceptable salt thereof. In a further embodiment, the subject may have poor glycemic control.

In a fifth aspect, the present invention provides a method of improving cognition or cognitive function in a subject having an elevated level of one or more inflammatory biomarkers comprising administering to the subject an amount of less than 20 mg per day of COMPOUND I or a pharmaceutically acceptable salt thereof.

In a sixth aspect, the present invention provides a method of treating cognitive impairment in a subject having an elevated level of one or more inflammatory biomarkers comprising administering to the subject an amount of less than 20 mg per day of COMPOUND I or a pharmaceutically acceptable salt thereof.

In a seventh aspect, the present invention provides a method of improving cognition or cognitive function in a subject comprising administering to the subject an amount of less than 20 mg per day of COMPOUND I or a pharmaceutically acceptable salt thereof, wherein the level of one or more inflammatory biomarkers is reduced.

In an eighth aspect, the present invention provides a method of treating cognitive impairment in a subject comprising administering to the subject an amount of less than 20 mg per day of COMPOUND I or a pharmaceutically acceptable salt thereof, wherein the level of one or more inflammatory biomarkers is reduced.

In a ninth aspect, the present invention provides a method of improving cognition or cognitive function in a subject overexpressing RAGE comprising administering to the subject an amount of less than 20 mg per day of COMPOUND I or a pharmaceutically acceptable salt thereof.

In a tenth aspect, the present invention provides a method of treating cognitive impairment or improving cognition in a subject overexpressing RAGE comprising administering to the subject an amount of less than 20 mg per day of COMPOUND I or a pharmaceutically acceptable salt thereof.

In an eleventh aspect, the present invention provides a method of selecting a subject for treating cognitive impairment or improving cognition comprising administering to the subject an amount of less than 20 mg per day of COMPOUND I or a pharmaceutically acceptable salt thereof.

In a twelfth aspect, the present invention also provides a method of treating cognitive impairment or improving cognition in a subject comprising: selecting a subject for treatment having a diagnosis of having poor glycemic control, of having an elevated level of one or more RAGE ligands, of having an elevated level of one or more inflammatory biomarkers, of having an HbAc1 of greater than or equal to 6.5% and/or of overexpressing RAGE; and administering to the subject an amount of less than 20 mg per day of COMPOUND I or a pharmaceutically acceptable salt thereof.

In a thirteenth aspect, the present invention also provides a method of treating a subject having cognitive impairment comprising determining whether the subject has poor glycemic control, has an elevated level of one or more RAGE ligands, has an elevated level of one or more inflammatory biomarkers, has an HbA1c of greater than or equal to 6.5% and/or is overexpressing RAGE; and if the subject has poor glycemic control, has an elevated level of one or more RAGE ligands, has an elevated level of one or more inflammatory biomarkers, has an HbAc1 of greater than or equal to 6.5% and/or is overexpressing RAGE, then administering to the subject an amount of less than 20 mg per day of COMPOUND I or a pharmaceutically acceptable salt thereof.

In each of the above methods, the subject may exhibit treatment of cognitive impairment while exhibiting no significant or no pronounced improvement in glycemic control. For example, during a period of treatment a subject's HbA1c level may only be reduced by 0.5% or less relative to a baseline measurement or may not fall below 6.5%.

In an embodiment, cognitive impairment may include one or more of the following symptoms: impairment of short-term or long-term memory function, learning, mental flexibility, attention, executive function, spatial working memory, numerical working memory, picture recognition, word recognition, inspection time, mood, vigor, anger, hostility, confusion, and total mood disturbance.

In another embodiment, improving cognition or cognitive function may include improving one or more of a subject's short-term or long-term memory function, learning, mental flexibility, attention, executive function, spatial working memory, numerical working memory, picture recognition, word recognition, inspection time, or mood.

In another embodiment, the subject with cognitive impairment ranges from having mild cognitive impairment (MCI) to severe cognitive dysfunction.

In another embodiment, the subject with cognitive impairment has mild cognitive impairment (MCI) or has been diagnosed with MCI prior to treatment.

In another embodiment, the subject with cognitive impairment has an MMSE score of between 21-26 or between 19-27 prior to treatment, or has been diagnosed with an MMSE score of between 21-26 or between 19-27 prior to treatment.

In another embodiment, the subject with cognitive impairment has a CDR global score of 0.5 or 1 prior to treatment, or has been diagnosed with an CDR global score of 0.5 or 1 prior to treatment.

In another embodiment, the subject with cognitive impairment has an ADAS-cog14 score of greater than or equal to 10 prior to treatment, or has been diagnosed with an ADAS-cog14 score of greater than or equal to 10 prior to treatment.

In another embodiment, the subject with cognitive impairment may perform daily activities without assistance.

In another embodiment, the subject with cognitive impairment may suffer from dementia of Alzheimer's type, or mild Alzheimer's disease, or moderate Alzheimer's disease, or mild dementia of Alzheimer's type, or moderate dementia of Alzheimer's type, or vascular dementia.

In another embodiment, the subject with cognitive impairment may satisfy one or more criteria (such as ADAS-cog, MMSE, CDR-sb, ADCS-iADL) for mild cognitive impairment (MCI), or for mild Alzheimer's disease, or for moderate Alzheimer's disease. In a further embodiment, a subject with mild AD or having a diagnosis of mild AD has an MMSE between 21-26, an MMSE between 19-27, and/or an ADAS-cog14 of greater than or equal to 10.

In another embodiment, the subject is at least 50, 55, 60, 65, 70, 75, 80, 85, or 90 years old, or is between 50 to 70 years old, or is between 60-80 years old, or is between 65 to 85 years old.

In another embodiment, the subject is overexpressing RAGE.

In another embodiment, the subject has elevated levels of one or more inflammatory biomarkers. In an embodiment, the elevated inflammatory biomarker is soluble RAGE (sRAGE). In a further embodiment, the elevated inflammatory biomarker is a neuroinflammatory biomarker. In a further embodiment, the elevated inflammatory biomarker is one or more of IL2, IL6, IL12, IFNg, CD40L, MIP-1, TNFb, MCP-3, IL13, GM-CSF, FGF-2, Nfl, IL8, IL15, IL16, MCP-1, MDC, CRP, ICAM1, SAA, VACM1, endothelinl, and thrombomodulin. In a further embodiment, the elevated inflammatory biomarker is selected from the group consisting of IL2, IL6, IL12, IFNg, CD40L, MIP-1, and TNFb. In a further embodiment, the elevated inflammatory biomarker is selected from the group consisting of IL2, IL6, IL12, IFNg, and TNFb. In a further embodiment, the elevated inflammatory biomarker is one or more of IL2, IL6, IL12, IFNg, CD40L, MIP-1, and TNFb. In a further embodiment, the elevated inflammatory biomarker is one or more of IL2, IL6, IL12, IFNg, and TNFb.

In a further embodiment, the blood plasma level of one or more inflammatory biomarkers is reduced during treatment by at least 1%, 5%, 10%, 20%, 30%, 40%, 50%, 60%, 70%, or 80% relative to a baseline level such as prior to dosing. The period between measurements of the blood plasma level of one or more inflammatory biomarkers may be 1 week, 2 weeks, 3 weeks, 4 weeks, 2 months, 3 months, 6 months, 9 months, or 12 months. In a further embodiment, the blood plasma level of one or more of IL2, IL6, IL12, IFNg, CD40L, MIP-1, TNFb, MCP-3, IL13, GM-CSF, FGF-2, Nfl, IL8, IL15, IL16, MCP-1, MDC, CRP, ICAM1, SAA, VACM1, endothelinl, and thrombomodulin is reduced by at least 5%, 10%, 20%, 30%, 40%, 50%, 60%, 70%, or 80% relative to a baseline level such as prior to dosing. In a further embodiment, the blood plasma level of one or more of IL2, IL6, IL12, IFNg, CD40L, MIP-1, and TNFb is reduced by at least 5%, 10%, 20%, 30%, 40%, 50%, 60%, 70%, or 80% relative to a baseline level such as prior to dosing. In a further embodiment, the blood plasma level of one or more of IL2, IL6, IL12, IFNg, and TNFb is reduced by at least 5%, 10%, 20%, 30%, 40%, 50%, 60%, 70%, or 80% relative to a baseline level such as prior to dosing.

In another embodiment, the subject has an HbA1c level above 6.5%, 7.0%, 7.5%, 8.0%, 8.5%, or 9.0%, or between 6.5% and 9.5%, or at or above 6.5% and below 9.5%, 9.0%, 8.5%, 8.0%, 7.5% or 7.0%.

In another embodiment, the subject has type 1 diabetes or type 2 diabetes.

In another embodiment, the subject has had at least one, two, three, or four hypoglycemic events, severe hypoglycemic events, hyperglycemic events, and/or severe hyperglycemic events in their lifetime, or in the last 2, 4, 6, 8, 10, 20, or 30 years.

In another embodiment, the subject has a blood serum concentration of COMPOUND I or a pharmaceutically acceptable salt thereof between 0.2 ng/mL and 20 ng/mL, between 0.5 ng/mL and 18 ng/mL, between 1 ng/mL and 16 ng/mL, between 1.5 ng/mL and 14 ng/mL, or between 2.0 ng/mL and 12 ng/mL. The blood serum concentration of COMPOUND I or a pharmaceutically acceptable salt thereof may be measured after 1 week, 2 weeks, 3 weeks, or 4 weeks from commencement of treatment. The blood serum concentration may be a maximum trough concentration during a treatment period.

In another embodiment, the subject is administered sufficient amount of COMPOUND I or a pharmaceutically acceptable salt thereof over a period to maintain daily or weekly blood serum concentration or trough concentration of COMPOUND I or a pharmaceutically acceptable salt thereof between 0.2 ng/mL and 20 ng/mL, between 0.5 ng/mL and 18 ng/mL, between 1 ng/mL and 16 ng/mL, between 1.5 ng/mL and 14 ng/mL, or between 2.0 ng/mL and 12 ng/mL.

In another embodiment, the subject is administered sufficient amount of COMPOUND I or a pharmaceutically acceptable salt thereof over a period to maintain a daily blood serum trough concentration of COMPOUND I or a pharmaceutically acceptable salt thereof between 0.2 ng/mL and 20 ng/mL, between 0.5 ng/mL and 18 ng/mL, between 1 ng/mL and 16 ng/mL, between 1.5 ng/mL and 14 ng/mL, or between 2.0 ng/mL and 12 ng/mL, or maintain a daily blood serum trough concentration of COMPOUND I or a pharmaceutically acceptable salt thereof of no greater than 20 ng/mL, 18 ng/mL, 16 ng/mL, 14 ng/mL, or 12 ng/mL. The blood serum trough concentration of COMPOUND I or a pharmaceutically acceptable salt thereof may be measured after 1 week, 2 weeks, 3 weeks, or 4 weeks from commencement of treatment.

In another embodiment, the frequency of administration of a COMPOUND I or a pharmaceutically acceptable salt thereof is once a day, once every two days, three days, four days, five days, six days, or once a week and the amount administered is 1, 2, 3, 4, 5, 6, 7, 8, 9, 10, 11, 12, 13, 14, 15, 16, 17, 18, 19, or 20 mg. In an embodiment, between about 1 mg to about 19 mg is administered, or from about 1 mg to about 18 mg is administered, or from about 1 mg to about 17 mg is administered, or from about 1 mg to about 16 mg is administered, or from about 1 mg to about 15 mg is administered, or from about 1 mg to about 14 mg is administered, or from about 1 mg to about 13 mg is administered, or from about 1 mg to about 12 mg is administered, or from about 1 mg to about 11 mg is administered, or from about 1 mg to about 10 mg is administered, or from about 1 mg to about 9 mg is administered, or from about 1 mg to about 8 mg is administered, or from about 1 mg to about 7 mg is administered, or from about 1 mg to about 6 mg is administered, or from about 1 mg to about 5 mg is administered, or from about 1 mg to about 4 mg is administered, or from about 1 mg to about 3 mg is administered, or from about 1 mg to about 2 mg is administered, where the frequency of administration is once a day, once every two days, three days, four days, five days, six days, or once a week. In other embodiments, the dose is about 5 mg or about 4 mg or about 3 mg or about 2 mg, wherein the where the frequency of administration is once a day, once every two days, three days, four days, five days, six days, or once a week. In another embodiment, multiple discrete doses of COMPOUND I or a pharmaceutically acceptable salt thereof are administered during a period such that no more than 20 mg of COMPOUND I or a pharmaceutically acceptable salt thereof is administered within a 24 hour period.

In another embodiment, the treatment results in a reduction in the rate of deterioration of one or more measures of cognitive function, in no deterioration (or stable) in one or more measures of cognitive function, or in improvement in one or more measures of cognitive function. In a further embodiment, treatment is determined by at least one of the assessments (or a subgroup of criteria within an assessment) wherein the assessment is selected from Table A:

TABLE A
Neuropsychological Assessments

Alzheimer's Disease Assessment Scale-cognitive subscale (ADAS-cog, ADAS-cog
11 or ADAS-cog 14),
Clinical Dementia Rating Sum of Boxes (CDR-sb),
Alzheimer's Disease Cooperative Study Activities of Daily Living Scale (ADCS-
ADL), including the Instrumental Activities of Daily Living Scale (iADL)
and Basic Activities of Daily Living Scale (bADL) computed based on sub-
items from within the ADCS-ADL,
Neuropsychiatric Inventory (NPI),
Mini-Mental State Examination (MMSE),
Mini-Cog, AD8 Dementia Screening Interview,
Informant Questionnaire on Cognitive Decline in the Elderly (IQCODE),
Montreal Cognitive Assessment (MoCA),
Direct Assessment of Functional Status (DAFS),
Everyday Problems Test (EPT),
Financial Capacity Instrument (FCI),
Management of Everyday Technology Assessment (META),
Timed Instrumental Activities of Daily Living (TIADL),
Alzheimer's Disease Cooperative Study/Activities of Daily Living Inventory
adapted for patients with mild cognitive impairment - 18 items (ADCS-
MCI-ADL-18),
Alzheimer's Disease Cooperative Study/Activities of Daily Living scale adapted
for patients with mild cognitive impairment - 24 items (ADCS-MCI-ADL-
24),
Activities of Daily Living-Prevention Instrument (ADL-PI),
Self-report Instrumental Activities of Daily Living (S-IADL),
Inspection time (IT),
The Profile of Mood States (POMS),
Raven's Progressive Matrices,
UWIST Mood Adjective Checklist (UMACL),
Spielberger State-Trait Anxiety Inventory (STAI),
Perceived Stress Scale (PSS),
the Amsterdam-Instrumental Activity of Daily Living (Amsterdam-IADL),
Dementia Quality of Life assessments (DEMQOL or DEMQOL-Proxy),
the Functional Activities Questionnaire (FAQ)

In an embodiment, the treatment results in a reduction in the rate of deterioration of one or more measures of cognitive function, in no deterioration (or stable) in one or more measures of cognitive function, or in improvement in one or more measures of cognitive function, wherein the measure of cognitive function is one or more of ADAS-cog14, CDR-sb, FAQ, Amsterdam-IADL, MMSE and/or eGFR and the period for measure is 3, 6, 9, 12, 15 or 18 months. In another embodiment, the treatment results in a reduction in the rate of deterioration of one or more measures of cognitive function, in no deterioration (or stable) in one or more measures of cognitive function, or in improvement in one or more measures of cognitive function, wherein the measure of cognitive function is ADAS-cog14 and/or CDR-sb and the period for measure is 3, 6, 9, 12, 15 or 18 months.

In another embodiment, the treatment results in a statistically significant reduction in the rate of deterioration of one or more measures of cognitive function, in no deterioration in one or more measures of cognitive function, or in improvement in one or more measures of cognitive function in a subject relative to a placebo group. In a further embodiment, the measure of statistical significance is a p value of less than or equal to 0.08, 0.07, 0.06, 0.05, 0.04, 0.03, 0.02, or 0.01 or is less than 0.08, 0.07, 0.06, 0.05, 0.04, 0.03, 0.02, or 0.01. Statistically significant change may be measured after 3, 6, 9, 12, 15, or 18 months of treatment.

In another embodiment, the treatment results in a statistically significant reduction in the levels of one or more inflammatory biomarkers in the subject. In a further embodiment, the one or more inflammatory biomarker is selected from the group consisting of IL2, IL6, IL12, IFNg, CD40L, MIP-1, TNFb, MCP-3, IL13, GM-CSF, FGF-2, Nfl, IL8, IL15, IL16, MCP-1, and MDC. In a further embodiment, the one or more inflammatory biomarker is selected from the group consisting of CRP, ICAM1, SAA, VACM1, endothelinl, and thrombomodulin. In a further embodiment, the one or more inflammatory biomarker is a neuroinflammatory biomarker. In a further embodiment, the one or more inflammatory biomarker is selected from the group consisting of IL2, IL6, IL12, IFNg, CD40L, MIP-1, and TNFb. In a further embodiment, the level of elevated one or more inflammatory biomarker selected from the group consisting of IL2, IL6, IL12, IFNg, and TNFb is reduced. Statistically significant change may be measured after 3, 6, 9, 12, 15, or 18 months of treatment.

In another embodiment, the dosage or blood level of COMPOUND I or a pharmaceutically acceptable salt thereof and administration may be sufficient for inhibition of the biological function of RAGE at a sufficient level for sufficient time to decrease or improve or treat cognitive impairment in a subject having poor glycemic control.

In another embodiment, the elevated RAGE ligand may be one or more of advanced glycation endproducts (AGEs), S100, calgranulin, EN-RAGE, 6-amyloid (including Abeta (1-40) and Abeta (1-42)), CML (N^{ε}-Carboxymethyl lysine), high mobility group protein B1 (HMGB1), and amphoterin. In a further embodiment, an elevated level of a RAGE ligand is the upper quarter of a plasma range in humans or relative to a healthy control in humans. In a further embodiment, the level of RAGE ligand is measured directly from the plasma, recovered from the plasma matrix after diluting the plasma sample in a formulated buffer, or associated with the remaining cellular pellet, or a collection of all of these. In a further embodiment, the elevated RAGE ligand is Abeta (1-40). In a further embodiment, the elevated RAGE ligand is Abeta (1-40) and the plasma level of Abeta (1-40) is above 160 pg/mL, above 165 pg/mL, above 170 pg/mL, above 175 pg/mL, above 180 pg/mL, above 185 pg/mL, above 190 pg/mL, above 195 pg/mL, above 200 pg/mL, above 210 pg/mL, above 225 pg/mL, above 250 pg/mL, or above 300 pg/mL.

In any of the embodiments described herein, a treated subject may exhibit a benefit in one or more measures of cognition as described above while having no significant or pronounced improvement in glycemic control. In embodiment, during a period of treatment, a subject's HbA1c level may only be reduced by 1.0%, 0.9%, 0.8%, 0.6%, 0.5%, 0.4%, 0.3%, 0.2%, or 0.1% percent points or less relative to a baseline measurement, or a subject's HbA1c may increase during treatment, or a subject's HbA1c may not fall below 5.7%, or 6.0%, or 6.5%, or 7.0% during treatment, or a subject's non-fasting glucose may only be reduced by 25 mg/di, or 20 mg/dL, or 15 mg/dL, or 10 mg/dL, or 5 mg/dL or less, or a subject's non-fasting glucose may increase during treatment.

The methods of treatment disclosed herein can include a first step of selecting a subject for treatment. In some embodiments, the subject is selected for treatment when the subject exhibits one or more of the clinical symptoms of a cognitive disorder or loss of cognitive function. In another embodiment, the subject is selected for treatment when the subject is determined to be overexpressing RAGE, have an elevated level of a RAGE ligand, or an elevated level of one or more inflammatory biomarkers.

In some embodiments, the subject is selected for treatment when a combination of clinical symptoms and biochemical markers are identified. For example, subjects can be selected for treatment based on a combination of two or more of the following: a clinical identification of one or more of the symptoms of a cognitive disorder or loss of cognitive function, a diagnosis of poor glycemic control, an HbA1c level of at least 6.5%, having had at least one, two, three, or four hypoglycemic events, severe hypoglycemic events, hyperglycemic events, and/or severe hyperglycemic events in their lifetime, or in the last 2, 4, 6, 8, 10, 20, or 30 years, a diagnosis of diabetes, a determination of having an elevated level of a RAGE ligand, a determination of having an elevated level of one or more inflammatory biomarkers, or a determination of overexpressing RAGE.

In some embodiments, the methods of treatment may further comprise the step of determining whether a subject has one or more of the symptoms of a cognitive disorder or loss of cognitive function, had at least one, two, three, or four hypoglycemic events, severe hypoglycemic events, hyperglycemic events, and/or severe hyperglycemic events in their lifetime, or in the last 2, 4, 6, 8, 10, 20, or 30 years by requesting the information from the subject or conducting a scale to determine if the subject has one or more of the symptoms of a cognitive disorder or loss of cognitive function, had at least one, two, three, or four hypoglycemic events, severe hypoglycemic events, hyperglycemic events, and/or severe hyperglycemic events in their lifetime, or in the last 2, 4, 6, 8, 10, 20, or 30 years, and if so, the method further comprises administering to the subject a therapeutically effective amount of COMPOUND I or a pharmaceutically acceptable salt thereof.

In some embodiments, the methods of treatment may further comprise the step of determining whether a subject has poor glycemic control, an HbA1c level of at least 6.5%, diabetes, an elevated level of a RAGE ligand, elevated level of one or more inflammatory biomarkers, or is overexpressing RAGE by obtaining or having obtained a biological sample from the subject and performing or having performed a bodily fluid test on the biological sample to determine if the subject has poor glycemic control, an HbA1c level of at least 6.5%, diabetes, an elevated level of a RAGE ligand, elevated level of one or more inflammatory biomarkers, or is overexpressing RAGE. If the subject is determined to have poor glycemic control, an HbA1c level of at least 6.5%, diabetes, an elevated level of a RAGE ligand, elevated level of one or more inflammatory biomarkers, or is overexpressing RAGE, the method further comprises administering to the subject a therapeutically effective amount of COMPOUND I or a pharmaceutically acceptable salt thereof.

In some embodiments, the methods of treatment may further comprise the step of determining the blood serum concentration of COMPOUND I or a pharmaceutically acceptable salt thereof by obtaining or having obtained a biological sample from the subject and performing or having performed a bodily fluid test on the biological sample to determine. In a further embodiment, if blood serum concentration of COMPOUND I or a pharmaceutically acceptable salt thereof is above 20 ng/mL, then the method further comprises reducing the dosage amount and/or frequency of dosing. In a further embodiment, if blood serum concentration of COMPOUND I or a pharmaceutically acceptable salt thereof is below 0.2 ng/mL, then the method further comprises increasing the dosage amount and/or frequency of dosing.

The methods of treatment disclosed herein may include a first step of selecting a subject for treatment having a diagnosis of having poor glycemic control, of having an elevated level of one or more RAGE ligands, of having an elevated level of one or more inflammatory biomarkers, of having an HbAc1 of greater than or equal to 6.5% and/or of overexpressing RAGE; and second step of administering to the subject an amount of less than 20 mg per day of COMPOUND I or a pharmaceutically acceptable salt thereof.

The methods of treatment disclosed herein may include a first step of determining whether the subject has poor glycemic control, has an elevated level of one or more RAGE ligands, has an elevated level of one or more inflammatory biomarkers, has an HbAc1 of greater than or equal to 6.5% and/or is overexpressing RAGE; and if the subject has poor glycemic control, has an elevated level of one or more RAGE ligands, has an elevated level of one or more inflammatory biomarkers, has an HbAc1 of greater than or equal to 6.5% and/or is overexpressing RAGE, then a second step of administering to the subject an amount of less than 20 mg per day of COMPOUND I or a pharmaceutically acceptable salt thereof. In any of the preceding methods, the method may further comprise obtaining or having obtained biological samples over a period of time from the subject and performing or having performed a bodily fluid test on the biological samples to determine whether the level of one or more biochemical markers are increasing or decreasing, and if the level of one or more biochemical markers are not trending in the desired direction then administering a greater dose of a compound of COMPOUND I or a pharmaceutically acceptable salt thereof. For example, if the level of one or more inflammatory biomarkers is not reduced by at least 1%, 5%, 10%, 20%, 30%, 40%, 50%, 60%, 70%, or 80% relative to a baseline level (such as prior to dosing), then the method comprises administering a greater dose and/or a more frequent dose of COMPOUND I or a pharmaceutically acceptable salt thereof. The period between measurements of an inflammatory biomarker may be 1, 2, 3, or 4 weeks, or 3, 6, 9, 12, 15, or 18 months. In an embodiment, the measured inflammatory biomarker is a neuroinflammatory biomarker. In a further embodiment, the measured inflammatory biomarker is selected from the group consisting of IL2, IL6, IL12, IFNg, CD40L, MIP-1, and TNFb. In a further embodiment, the measured inflammatory biomarker is selected from the group consisting of IL2, IL6, IL12, IFNg, and TNFb.

The period between collection of biological samples from a subject may be 1 week, 2 weeks, 3 weeks, 4 weeks, 2 months, 3 months, 6 months, 9 months, or 12 months and COMPOUND I or a pharmaceutically acceptable salt thereof may be administered during this period.

Biomarkers that may predict a response to therapy with a RAGE antagonist such as COMPOUND I or a pharmaceutically acceptable salt thereof in subjects having a cognitive disorder are provided herein. The level of HbA1c prior to treatment (such as above 5.7%, above 6.0%, or above 6.5%) may also predict a response to therapy with a RAGE antagonist such as COMPOUND I or a pharmaceutically acceptable salt thereof in subjects having a cognitive disorder.

Methods are also provided for the diagnosis and monitoring of treatment of a cognitive disorder based on detection of certain biomarkers in samples from patients who have, or are suspected of having, a cognitive disorder. Further, expression of one or more such biomarkers can be used to distinguish subjects that respond favorably to treatment with a RAGE antagonist such as COMPOUND I or a pharmaceutically acceptable salt thereof from subjects that don't respond to therapy and from subjects that have a partial response to therapy.

Analysis of levels of expression and/or activity of gene products correlated with a subject's response to treatment with COMPOUND I has led to the identification of certain biomarker signatures. For example, the present invention provides methods for evaluation of expression level of one, two, three, four, five, six, seven, eight, nine, ten, fifteen, twenty, twenty-five, thirty, thirty-five, or more genes from Table B.

TABLE B
Human Cytokines and Chemokines

EGF, Eotaxin, G-CSF, GM-CSF, IFNα2, IFNγ, IL-10, IL-12P40, IL-12P70, IL-13, IL-
15, IL-17A, IL-1RA, IL-1α, IL-1β, IL-2, IL-3, IL-4, IL-5, IL-6, IL-7, IL-8, IP-10, MCP-
1, MIP-1α, MIP-1β, RANTES, TNFα, TNFβ, VEGF, FGF-2, TGF-α, FIT-3L,
Fractalkine, GRO, MCP-3, MDC, PDGF-AA, PDGF-AB/BB, sCD40L, and IL-9

Table B lists biomarker proteins that may be differentially expressed prior to or during treatment by complete responders compared to partial responders and non-responders. In an embodiment, the biomarker proteins that may be differentially expressed prior to or during treatment by complete responders compared to partial responders and non-responders are one or more of the biomarkers IL2, IL6, IL12, IFNg, CD40L, MIP-1, and TNFb. In another embodiment, the biomarker proteins that may be differentially expressed prior to or during treatment by complete responders compared to partial responders and non-responders are one or more of the biomarkers IL2, IL6, IL12, IFNg, and TNFb.

In some embodiments, methods of the present disclosure can be used to determine the responsiveness of a subject to treatment with a RAGE antagonist such as COMPOUND I or a pharmaceutically acceptable salt thereof, wherein a statistically significant difference in the amount, e.g., expression, and/or activity of a marker disclosed herein relative to a reference, e.g., a median value for a patient population, a median value for a population of healthy subjects, a median value for a population of non-responders or partial responders, in a subjects sample, then the more likely the disease is to respond to a RAGE antagonist such as COMPOUND I or a pharmaceutically acceptable salt thereof.

In an embodiment, the disclosure provides a method of, or assay for, identifying a subject having a cognitive disorder as having an increased or decreased likelihood to respond to a treatment that comprises administering a RAGE antagonist such as COMPOUND I or a pharmaceutically acceptable salt thereof, the method comprising:

(1) acquiring a sample from the subject;

(2) determining a level of one or more (e.g., 2, 3, 4, 5, 6, 7, 8, 9, 10, 15 or more) inflammatory markers such as those listed in Table A; and

(3) comparing the determined level of one or more markers to a reference level; wherein a difference, e.g., statistically significant difference in the determined level to the reference level is predictive of the subject's responsiveness to a RAGE antagonist such as COMPOUND I or a pharmaceutically acceptable salt thereof; and

(4) identifying the subject as a complete responder, partial responder or non-responder to therapy.

The methods provided herein may be useful for identifying subjects that are likely to respond to a RAGE antagonist such as COMPOUND I or a pharmaceutically acceptable salt thereof prior to initiation of such treatment (e.g., pre-therapy) or early in the therapeutic regimen. In some embodiments, expression or activity of biomarkers is measured in a subject at least 2 weeks, at least 1 month, at least 3 months, or at least 6 months, prior to initiation of therapy. In other embodiments, the expression or activity of biomarkers is determined after initiation of therapy such as after 1, 2, 3, 6, 9, 12, 15, or 18 months of treatment. The methods described herein can also be used to monitor a positive response of a subject to a RAGE antagonist such as COMPOUND I or a pharmaceutically acceptable salt thereof. Such methods are useful for early detection of tolerance to therapy or to predict whether a subject will progress. In such embodiments, the expression or activity of biomarkers is determined e.g., at least every week, at least every 2 weeks, at least every month, at least every 2 months, at least every 3 months, at least every 4 months, at least every 5 months, at least every 6 months, at least every 7 months, at least every 8 months, at least every 9 months, at least every 10 months, at least every 11 months, at least every year. It is also contemplated that expression or activity of the biomarkers is at irregular intervals e.g., biomarkers can be detected in a subject at 3 months of treatment, at 6 months of treatment, and at 7 months of treatment. Thus, in some embodiments, the expression or activity of the biomarkers is determined when deemed necessary by the skilled physician monitoring treatment of the subject.

### Definitions

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “about” and “approximately” shall generally mean an acceptable degree of error or variation for the quantity measured given the nature or precision of the measurements. Typically, degrees of error or variation are within 20 percent (%), preferably within 10%, and more preferably within 5% of a given value or range of values. Numerical quantities given herein are approximate unless stated otherwise, meaning that the term “about” or “approximately” can be inferred when not expressly stated.

By percent by weight it is meant that a particular weight of one ingredient in a composition is divided by the total weight of all of the ingredients in that composition. Percent by weight may be used interchangeably and means approximately the same as weight/weight percent or % (weight/weight) or percent by mass or mass percent.

It is further noted that, as used in this specification, the singular forms “a,” “an,” and “the” include plural referents unless expressly and unequivocally limited to one referent.

COMPOUND I refers to [3-(4-{2-butyl-1-[4-(4-chloro-phenoxy)-phenyl]-1H-imidazol-4-yl}-phenoxy)-propyl]-diethyl amine. COMPOUND I is the subject matter of U.S. Pat. Nos. 7,361,678 and 7,884,219.

Various salts and isomers of COMPOUND I can be used. The term “salts” can include acid addition salts or addition salts of free bases. Examples of acids which may be employed to form pharmaceutically acceptable acid addition salts include inorganic acids such as hydrochloric, sulfuric, or phosphoric acid, and organic acids such as acetic, maleic, succinic, or citric acid, etc. All of these salts (or other similar salts) may be prepared by conventional means. The nature of the salt is not critical, provided that it is non-toxic and does not substantially interfere with the desired pharmacological activity. A preferred salt for the method of the present invention is the hydrochloride salt.

The phrase “pharmaceutically acceptable”, as used in connection with compositions of the invention, refers to molecular entities and other ingredients of such compositions that are physiologically tolerable and do not typically produce untoward reactions (toxicity or side effects) when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. Berge, et al. Journal of Pharmaceutical Science, Vol. 66(1), pp. 1-19 (1977).

The term “carrier” applied to pharmaceutical compositions of the invention refers to a diluent, excipient, or vehicle with which an active compound (e.g., an 1-aminocyclohexane derivative) is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18th Edition.

The term “subject” or “subject in need thereof” as used herein refers to a mammal. In an embodiment, the subject is a human. In a further embodiment, the subject does not use insulin.

Non-limiting examples of RAGE ligands include Advanced glycation end products (AGEs); Members of the 5100/calgranulin family (e.g., calgranulin C (also known as ENRAGE and S100A12), S100A1, S100A4, S100A11, S100A13, S100B, and S100P); Amyloid-ß-peptide (Aß), as for example Aß 1-40 peptide Amyloid-ß globulomers; as for example Aß₁₋₄₂, Aß₁₂₋₄₂, Aß₂₀₋₄₂ globulomers, amphoterin, CML, and HMGB1.

Mild or moderate Alzheimer's disease can be diagnostically assessed as “probable Alzheimer's” according to the National Institute of Neurological and Communicative Disorders and Stroke/the Alzheimer's Disease and Related Disorders Associations (NINCDS-ADRDA) criteria.

The diagnosis of mild or moderate AD is well within the purview of the ordinary skilled physician or trained assistant using standard criteria, including the clinical assessment scales disclosed above and below. By way of example, the following numerical ranges on the standardized Mini-Mental State Examination (MMSE; 0-30 scale) have been used to diagnose mild-to-moderate, moderate, and moderate-to-severe Alzheimer's.

Mild-to-moderate Alzheimer's disease has been diagnosed as determined by MMSE scores of 10 to 22, and also from 10-26. Severe Alzheimer's has been diagnosed in subjects having MMSE scores of less than 10.

Accordingly, a diagnosis of “mild” Alzheimer's disease could be made for subjects having the higher scores within the above-described ranges, e.g., about 21 to 26 on the MMSE. A diagnosis of “moderate” Alzheimer's disease could be made for subjects having scores within the range of 10 to 20 on the MMSE.

It should be noted that the MMSE scale is not the only way to diagnose mild Alzheimer's disease, but represents a convenience. Nor should the claims be construed as requiring the step of “grading” a subject on the MMSE scale to be performed. In an embodiment, a subject having mild Alzheimer's disease is a patient who would score 21 or higher if the patient were scored according to MMSE scale. If a different scale were to be used, “mild” Alzheimer's disease would be defined as a diagnosis of Alzheimer's disease or probable Alzheimer's disease which is made based on a score that clearly does not overlap with the score range for moderate-to-severe Alzheimer's disease established for the same scale.

In an embodiment, mild Alzheimer's disease is defined as individuals having an ADAS-cog score of less than or equal to 23.

The term “treatment” as used herein, refers to the full spectrum of treatments for a given condition or disorder from which a subject is suffering, including alleviation or amelioration of one or more of the symptoms resulting from that disorder, to the delaying of the onset or progression of the disorder.

The term “treat” is used herein to mean to relieve or alleviate at least one symptom of a disease or condition in a subject. For example, the term “treat” may mean to relieve or alleviate cognitive impairment (such as impairment of memory and/or orientation) or impairment of global functioning (activities of daily living) and/or slow down or reverse the progressive deterioration in activities of daily living or cognitive impairment in individuals having a cognitive impairment.

Within the meaning of the present invention, the term “treat” may also mean delay of the progression of a disease in the patients presenting with additional symptoms associated with cognitive impairment, such as but not limited to those identified using one or more of the ADAS-cog, the MMSE, the ADCS-ADL criteria, the CDR-sb, or the NPI total criteria, defined above. The term “delay the progression” is used herein to mean slower than expected development or continuance or aggravation of a disease in a subject compared to an untreated subject. This can be determined for Alzheimer's disease, for example, by obtaining slower than expected deterioration in measures such as cognitive performance in treated patients, compared with those measures in untreated patients (who represent the expected progression of the disease). Cognitive performance can be measured using, e.g., the Alzheimer's Disease Assessment Scale (ADAS-cog), or the Alzheimer's Disease Cooperative Study-Activities of Daily Living (ADCS-ADL). For example, the typical disease progression in subjects with mild Alzheimer's disease is an increase of about 1 to about 3 points on the ADAS-cog over a time period of about 6 months. Treatment may also be measured by an improvement or stabilization of one or more measures of the following symptoms of cognitive impairment: impairment of short-term or long-term memory function, learning, mental flexibility, attention, executive function, spatial working memory, numerical working memory, picture recognition, word recognition, inspection time, mood, vigor, anger, hostility, confusion, and total mood disturbance. However, disease progression is highly individualized, and also depends on factors such as the initial condition of the patient.

The term “therapeutically effective amount” is used herein to mean an amount or dose of COMPOUND I that is effective to ameliorate or delay a symptom. Alternatively, a therapeutically effective amount is sufficient to cause an improvement in a clinically significant condition or parameter (according to the attending physician employing one or more of the foregoing sets of criteria) associated with cognitive impairment in an individual in need thereof. In still another embodiment, a therapeutically effective amount is used herein to denote the amount of COMPOUND I or a pharmaceutically acceptable salt thereof that will elicit the therapeutic response of a subject that is being sought. In an embodiment, in any of the methods of the present invention, the amount of COMPOUND I or a pharmaceutically acceptable salt administered may be a therapeutically effective amount.

As used herein, the terms “poor glycemic control” or “subject having poor glycemic control” refer to a subject having persistently elevated blood glucose levels, glycosylated hemoglobin levels and/or refers to a subject having had at least one hypoglycemic event, severe hypoglycemic event, hyperglycemic event, and/or severe hyperglycemic event in their lifetime. In an embodiment, a subject having poor glycemic control may have an HbA1c level of 5.7% or above, or 6.0% or above, or 6.5% or above, or 7.0% or above, or 8.0% or above, or 9.0% or above, or may have a persistent blood glucose level of greater than 90 mg/dL, or 100 mg/dL, or 125 mg/dL, or 150 mg/dL or above. The persistent blood glucose level may be fasting or non-fasting. In an embodiment, a subject having poor glycemic control has been diagnosed with type 2 or type 1 diabetes.

As used herein, an “improvement in glycemic control” for a subject having poor glycemic control may be measured by a reduction in blood glucose levels (fasting or non-fasting), a reduction in glycosylated hemoglobin levels, and/or a reduction in the number of hypoglycemic, severe hypoglycemic, hyperglycemic, and/or severe hyperglycemic events during a period of treatment.

As used herein, the terms “blood glucose level”, “blood sugar level”, “plasma glucose level” and “blood sugar concentration” refer to the amount of glucose present in the blood of a subject, and these terms may be used interchangeably. Blood glucose levels are typically measured in units of mg/dL or mmol/L.

As used herein, the term “hypoglycemia” refers to a blood glucose level below a normal level for a subject. In a human, hypoglycemia may be defined as a blood glucose level of less than 70 mg/dL. In an embodiment, hypoglycemia in a human is a blood glucose level of less than 70 mg/dL and greater than or equal to 54 mg/dL.

As used herein, the term “severe hypoglycemia” refers to a blood glucose level significantly below a normal level for a subject. In a human, severe hypoglycemia may be defined as a blood glucose level of less than 54 mg/dL.

As used herein, the term “hypoglycemic event” refers to a blood glucose level below normal level for a subject for a period of time. In an embodiment, a hypoglycemic event may occur upon a single measure of blood glucose level below normal through self-monitoring blood glucose (SMBG). In other embodiments, where blood glucose levels are continuously monitored, a hypoglycemic event may occur over a period of time such as where the blood glucose level is continuously below normal for at least 1, 2, 3, 4, 5, 6, 7, 9, 10, 12, 15, 20, 25, 30, 60, or 120 minutes. In an embodiment, in a human a hypoglycemic event may be defined as a blood glucose level of less than 70 mg/dL for a period of time or a single SMBG measurement. In another embodiment, in a human a hypoglycemic event may be defined as a blood glucose level of less than 70 mg/dL and greater than or equal to 54 mg/dL for a period of time or a single SMBG measurement.

As used herein, the term “severe hypoglycemic event” refers to a blood glucose level significantly below a normal level for a subject for a period of time. In an embodiment, a severe hypoglycemic event may result in the subject requiring external help to effect recovery including being hospitalized. In another embodiment, a severe hypoglycemic event may occur upon a single measure of blood glucose level significantly below normal through self-monitoring blood glucose (SMBG). In other embodiments, where blood glucose levels are continuously monitored, a severe hypoglycemic event may occur over a period of time such as where the blood glucose level is continuously below normal for at least 1, 2, 3, 4, 5, 6, 7, 9, 10, 12, 15, 20, 25, 30, 60, or 120 minutes. In an embodiment, in a human a severe hypoglycemic event may be defined as a blood glucose level of less than 54 mg/dL for a period of time or a single SMBG measurement.

As used herein, the term “hyperglycemia” refers to a blood glucose level above the normal level in a subject. In a human, hyperglycemia may be defined as a blood glucose level of greater than 180 mg/dL. In an embodiment, hyperglycemia in a human is a blood glucose level of greater than 180 mg/dL and less than or equal to 250 mg/dL.

As used herein, the term “severe hyperglycemia” refers to a blood glucose level significantly above the normal level in a subject. In a human, severe hyperglycemia may be defined as a blood glucose level of greater than 250 mg/dL.

As used herein, the term “hyperglycemic event” refers to a blood glucose level above normal level for a subject for a period of time. In an embodiment, a hyperglycemic event may occur upon a single measure of blood glucose level below normal through self-monitoring blood glucose (SMBG). In other embodiments, where blood glucose levels are continuously monitored, a hyperglycemic event may occur over a period of time such as where the blood glucose level is continuously above normal for at least 1, 2, 3, 4, 5, 6, 7, 9, 10, 12, 15, 20, 25, 30, 60, or 120 minutes. In an embodiment, in a human a hyperglycemic event may be defined as a blood glucose level of greater than 180 mg/dL for a period of time or a single SMBG measurement. In another embodiment, in a human a hyperglycemic event may be defined as a blood glucose level of greater than 180 mg/dL and less than or equal to 250 mg/dL for a period of time or a single SMBG measurement.

As used herein, the term “severe hyperglycemic event” refers to a blood glucose level significantly above a normal level for a subject for a period of time. In an embodiment, a severe hypoglycemic event may result in the subject requiring external help to effect recovery including being hospitalized. In another embodiment, a severe hyperglycemic event may occur upon a single measure of blood glucose level significantly above normal through self-monitoring blood glucose (SMBG). In other embodiments, where blood glucose levels are continuously monitored, a severe hyperglycemic event may occur over a period of time such as where the blood glucose level is continuously above normal for at least 1, 2, 3, 4, 5, 6, 7, 9, 10, 12, 15, 20, 25, 30, 60, or 120 minutes. In an embodiment, in a human a severe hyperglycemic event may be defined as a blood glucose level of greater than 250 mg/dL for a period of time or a single SMBG measurement.

A “biomarker” or “marker” is a gene, mRNA, or protein that undergoes alterations in expression that may be associated with inflammation, neuroinflammation, poor glycemic control, a cognitive disorder, or in response to treatment. The alteration can be in amount and/or activity in a biological sample (e.g., a blood, plasma, or a serum sample) obtained from a subject having a cognitive disorder and/or poor glycemic control as compared to its amount and/or activity, in a sample obtained from a baseline or prior value for the subject, the subject at a different time interval, an average or median value for a cancer patient population, a healthy control, or a healthy subject population (e.g., a control); such alterations in expression and/or activity are associated with of the responsiveness of a subject having a cognitive disorder and/or poor glycemic control to therapy with COMPOUND I. For example, a marker of the invention which is predictive of responsiveness to COMPOUND I can have an altered expression level, protein level, or protein activity, in a biological sample obtained from a subject having, or suspected of having, a cognitive disorder as compared to a biological sample obtained from a control subject.

A “nucleic acid marker” or “nucleic acid biomarker” is a nucleic acid (e.g., DNA, mRNA, cDNA) encoded by or corresponding to a marker as described herein.

A “marker protein” is a protein encoded by or corresponding to a nucleic acid marker. A marker protein comprises the entire or a partial sequence of a protein encoded by a nucleic acid marker, or a fragment thereof.

An “overexpression” or “significantly higher level of expression” of products refers to an expression level or copy number in a test sample that is greater than the standard error of the assay employed to assess the level of expression. In embodiments, the overexpression can be at least two, at least three, at least four, at least five, or at least ten or more times the expression level of the gene in a control sample or the average expression level of gene products in several control samples.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

The amount of a biomarker, e.g., expression of gene products (e.g., one or more the biomarkers described herein), in a subject is “significantly” higher or lower than the normal amount of a marker, if the amount of the marker is greater or less, respectively, than the normal level by an amount greater than the standard error of the assay employed to assess amount, or at least two, three, four, five, ten or more times that amount. Alternatively, the amount of the marker in the subject can be considered “significantly” higher or lower than the normal amount if the amount is at least about 1.5, two, at least about three, at least about four, or at least about five times, higher or lower, respectively, than the normal amount of the marker.

Formulations

The term “pharmaceutical composition” is used herein to denote a composition that may be administered to a mammalian host, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

The pharmaceutical compositions containing a compound of the invention may comprise less than 20 mg of COMPOUND I or a pharmaceutically acceptable salt thereof. In embodiment, a pharmaceutical composition may comprise 1, 2, 3, 4, 5, 6, 7, 8, 9, 10, 11, 12, 13, 14, 15, 16, 17, 18, or 19 mg of COMPOUND I or a pharmaceutically acceptable salt thereof and a pharmaceutically acceptable carrier. In another embodiment, a pharmaceutical composition may comprise between 1 to 20 mg, 1 to 7 mg, 2 to 8 mg, 3 to 8 mg, 4 to 8 mg, or 4 to 7 mg of COMPOUND I or a pharmaceutically acceptable salt thereof and a pharmaceutically acceptable carrier.

The pharmaceutical compositions containing a compound of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions or suspensions, lotions, eye ointments and eye or ear drops, impregnated dressings and aerosols etc., containing the compounds of the invention are contemplated. These topical formulations may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 0.1% by weight up to about 99% by weight of the formulation. More usually they will form up to about 80% by weight of the formulation. For the purpose of this application, topical applications shall include mouth washes and gargles.

For administration by inhalation, the compound of the invention may be delivered in the form of an aerosol spray presentation from pressurized packs or a nebulizer, with the use of a suitable propellant, e.g. dichlorodifluoromethane, trichlorofluoromethane, dichlorotetrafluoroethane, tetrafluoroethane, heptafluoropropane, carbon dioxide or other suitable gas.

In the case of a pressurized aerosol, the dosage unit may be determined by providing a valve to deliver a metered amount. Capsules and cartridges of e.g. gelatin for use in an inhaler or insufflator may be formulated containing a powder mix of a compound of the invention and a suitable powder base such as lactose or starch.

In any of the preceding embodiments, the administration of COMPOUND I or a pharmaceutically acceptable salt thereof may additionally include treatment with an acetylcholinesterase inhibitor (AChEI). The AChEI may include donepezil hydrochloride, galantamine hydrochloride, rivastigmine tartrate, or tacrine hydrochloride. In still other embodiments, the administration of COMPOUND I or a pharmaceutically acceptable salt thereof may additionally include treatment with memantine. In some embodiments, the subjects may have been receiving treatment with an AChEI or memantine for at least four months prior to the administration of COMPOUND I or a pharmaceutically acceptable salt thereof.

Another embodiment of the present invention includes a pharmaceutical composition including between 1 mg and 20 mg of COMPOUND I or a pharmaceutically acceptable salt thereof, and an AChEI. In other embodiments, the pharmaceutical composition may include between 1 mg and 20 mg of COMPOUND I or a pharmaceutically acceptable salt thereof, and memantine. The AChEI may include donepezil hydrochloride, galantamine hydrochloride, rivastigmine tartrate, or tacrine hydrochloride. In some embodiments, the AChEI is donepezil hydrochloride present between 5 mg and 23 mg. In other embodiments, the AChEI is galantamine hydrochloride present between 16 mg and 24 mg. In yet other embodiments, the AChEI is rivastigmine tartrate present between 6 mg and 12 mg. In still other embodiments, the AChEI is tacrine hydrochloride present at 40 mg. In still other embodiments, memantine is present between 5 mg and 20 mg.

In any of the previous embodiments, a therapeutically effective amount of an acetylcholinesterase inhibitor (AChEI) or memantine may also be administered. For example, between 1 mg and 20 mg of COMPOUND I or a pharmaceutically acceptable salt thereof, and an AChEI may be administered. In other embodiments, between 1 mg and 20 mg of COMPOUND I or a pharmaceutically acceptable salt thereof, and memantine may be administered. The AChEI administered may include donepezil hydrochloride, galantamine hydrochloride, rivastigmine tartrate, or tacrine hydrochloride. In some embodiments, the AChEI administered is donepezil hydrochloride in an amount between 5 mg and 23 mg. In other embodiments, the AChEI administered is galantamine hydrochloride in an amount between 16 mg and 24 mg. In yet other embodiments, the AChEI administered is rivastigmine tartrate in an amount between 6 mg and 12 mg. In still other embodiments, the AChEI administered is tacrine hydrochloride in an amount at 40 mg. In still other embodiments, memantine is administered in an amount between 5 mg and 20 mg.

In some embodiments, the subjects may have been receiving treatment with an AChEI or memantine for at least one, two, three, or four months prior to the administration of COMPOUND I or a pharmaceutically acceptable salt thereof.

In a further embodiment of the present invention, COMPOUND I or a pharmaceutically acceptable salt thereof may be administered in combination with one or more further active substances selected from antidiabetic agents, antihyperlipidemic agents, antiobesity agents, antihypertensive agents and agents for the treatment of complications resulting from or associated with diabetes.

Suitable antidiabetic agents include insulin, GLP-1 (glucagon like peptide-1) derivatives, metformin, as well as orally active hypoglycemic agents.

Suitable orally active hypoglycemic agents may include imidazolines, sulfonylureas, biguanides, meglitinides, oxadiazolidinediones, thiazolidinediones, insulin sensitizers, α-glucosidase inhibitors, agents acting on the ATP-dependent potassium channel of the pancreatic f3-cells eg potassium channel openers, potassium channel openers, such as ormitiglinide, potassium channel blockers, GLP-1 agonists, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, and PPAR (peroxisome proliferator-activated receptor) and RXR (retinoid X receptor) agonists.

In another embodiment of the present invention, COMPOUND I or a pharmaceutically acceptable salt thereof is administered in combination with a sulphonylurea eg tolbutamide, chlorpropamide, tolazamide, glibenclamide, glipizide, glimepiride, glicazide or glyburide.

In another embodiment of the present invention, COMPOUND I or a pharmaceutically acceptable salt thereof is administered in combination with a biguanide eg metformin.

In another embodiment of the present invention, COMPOUND I or a pharmaceutically acceptable salt thereof is administered in combination with a meglitinide eg repaglinide or senaglinide/nateglinide.

In another embodiment of the present invention, COMPOUND I or a pharmaceutically acceptable salt thereof is administered in combination with a thiazolidinedione insulin sensitizer eg troglitazone, ciglitazone, pioglitazone, rosiglitazone, isaglitazone, darglitazone, englitazone.

In another embodiment of the present invention, COMPOUND I or a pharmaceutically acceptable salt thereof is administered in combination with an insulin sensitizer.

In another embodiment of the present invention, COMPOUND I or a pharmaceutically acceptable salt thereof is administered in combination with an α-glucosidase inhibitor e.g. voglibose, emiglitate, miglitol or acarbose.

In another embodiment of the present invention, COMPOUND I or a pharmaceutically acceptable salt thereof is administered in combination with a glycogen phosphorylase inhibitor.

In another embodiment of the present invention, COMPOUND I or a pharmaceutically acceptable salt thereof is administered in combination with insulin, insulin derivatives or insulin analogues. An insulin derivative may be selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl LysB²⁸ ProB²⁹ human insulin, B28-N^{ε}-palmitoyl Lys^{B28} Pro^{B29} human insulin, B30-N^{ε}-myristoyl-ThrB²⁹LysB³⁰ human insulin, B30-N^{ε}-palmitoyl-Thr^{B29}LysB³⁰ human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin, B29-N^{ε}-(ω-carboxyheptadecanoyl) human insulin, and B29-N^{ε}-myristoyl-des(B30) human insulin. The insulin analogue may be selected from the group consisting of: an analogue wherein position B28 is Asp, Lys, Leu, Val, or Ala and position B29 is Lys or Pro; and des(B28-B30), des(B27) or des(B30) human insulin. In another embodiment, the analogue is an analogue of human insulin wherein position B28 is Asp or Lys, and position B29 is Lys or Pro. In another embodiment, the analogue is des(B30) human insulin. In another embodiment, the insulin analogue is an analogue of human insulin wherein position B28 is Asp. In another embodiment, the analogue is an analogue wherein position B3 is Lys and position B29 is Glu or Asp.

Assays

Methods to measure biomarkers described herein, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), magnetic activated cell sorting (MACS), flow cytometry, laser scanning cytometry, hematology analyzer and assays based on a property of the protein including but not limited to DNA binding, ligand binding, or interaction with other protein partners.

The activity or level of a marker protein can also be detected and/or quantified by detecting or quantifying the expressed polypeptide. The polypeptide can be detected and quantified by any of a number of means well known to those of skill in the art. These can include analytic biochemical methods such as electrophoresis, capillary electrophoresis, high performance liquid chromatography (HPLC), thin layer chromatography (TLC), hypercliffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry and the like. A skilled artisan can readily adapt known protein/antibody detection methods for use in determining the expression level of one or more biomarkers in a serum sample.

Another agent for detecting a polypeptide is an antibody capable of binding to a polypeptide corresponding to a marker described herein, e.g., an antibody with a detectable label. Antibodies can be polyclonal or monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In another embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair (e.g., biotin-streptavidin)), or an antibody fragment (e.g., a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Proteins can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, one can immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, and magnetite.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

In another embodiment, the polypeptide is detected and/or quantified using Luminex® assay technology. The Luminex® assay separates tiny color-coded beads into e.g., distinct sets that are each coated with a reagent for a particular bioassay, allowing the capture and detection of specific analytes from a sample in a multiplex manner. The Luminex® assay technology can be compared to a multiplex ELISA assay using bead-based fluorescence cytometry to detect analytes such as biomarkers.

The disclosure also encompasses kits for detecting the presence of a polypeptide or nucleic acid corresponding to a marker described herein in a biological sample, e.g., a sample containing tissue, whole blood, serum, plasma, buccal scrape, saliva, cerebrospinal fluid, urine, stool, and bone marrow. Such kits may be used to determine if a subject is suffering from or is at increased risk of developing a cognitive disorder, or may be used to determine if a subject is responding or may likely respond to treatment. For example, the kit can comprise a labeled compound or agent capable of detecting a polypeptide or an mRNA encoding a polypeptide corresponding to a marker described herein in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

### EXAMPLES

### Example 1

The study was a randomized, double-blind, placebo-controlled trial in approximately 800 participants with probable mild AD (MMSE 21-26, CDR global 0.5-1), receiving stable standard of care therapy (acetylcholinesterase inhibitor and/or memantine; SoC) evaluating the efficacy and safety of 18 months of treatment with azeliragon 5 mg/day relative to placebo. The clinical trial design included two separate studies (A-Study and B-Study) operationally conducted under a single protocol. Each study was independently powered to evaluate efficacy with respect to co-primary endpoints of ADAS-cog and CDR-sb and each study was randomized independently. In each study participants were randomized 1:1 (site-based randomization) to azeliragon (5 mg/day) plus SoC or placebo plus SoC. Primary efficacy outcomes included co-primary endpoints of change from baseline in the ADAS-cog at Month 18 and change from baseline in the CDR-sb at Month 18. The key secondary endpoint was change from baseline in brain volumetrics (e.g., whole brain volume, ventricular volume, hippocampal volume) at Month 18.

A total of 880 patients were randomized (405 in the A-Study, 475 in the B-Study) at 88 sites in the US and Canada (A-Study) and 99 sites in the US, Canada, Ireland, UK, South Africa, Australia, and New Zealand (B-Study). At baseline, the A-Study participants were 53% male, mean age was 75 (SD 8.5) years, mean baseline MMSE score was 23.2 (SD 2.72) and mean baseline ADAS-cog score was 15.5 (SD 5.40). The B-study participants were 55% male, mean age of 75 (SD 8.6) years, and had a mean baseline MMSE score of 23.2 (SD 2.81) and mean baseline ADAS-cog score of 16.6 (SD 5.54). Statistical analysis of the study data included analyses that were planned in the protocol and statistical analysis plan and also exploratory and investigative analysis.

Both the A-Study and B-Study failed to achieve statistical significance for pre-specified analyses on the co-primary endpoints of ADAS-cog and CDR-sb.

### Example 2

While neither study met its primary outcome measures, a subgroup analysis produced the following results.

For this subgroup analysis, T2D was defined by patients having an HbA1c of 6.5% or more at baseline. Study entry criteria excluded patients with HbA1c>7.7%.

A total of 55 patients with T2D and a clinical diagnosis of mild AD (probable mild AD) were identified across both the A-Study and B-Study (n=22 placebo and n=33 azeliragon). Baseline characteristics were reasonably balanced between treatment groups for demography, background traits, and baseline characteristics, including characteristics of specific interest: sex, age, APOE status, and HbA1c.

Primary methodology used was the protocol-planned statistical model: the primary analysis used MMRM methodology with baseline as covariate, baseline stratum as a covariate, and subject as a random effect. The analysis population selection followed ICH E9 recommendations for randomization support (criteria are based on pre-randomization data and are applied to all patients in the study).

Safety analysis revealed no trends or treatment-emergent adverse events (TEAEs) of concern among patients treated with azeliragon relative to placebo in this subgroup.

Efficacy subgroup analysis results are presented in FIGS. 1-3. All p-values presented in FIGS. 1-3 are nominal, since the primary analysis of the studies was negative.

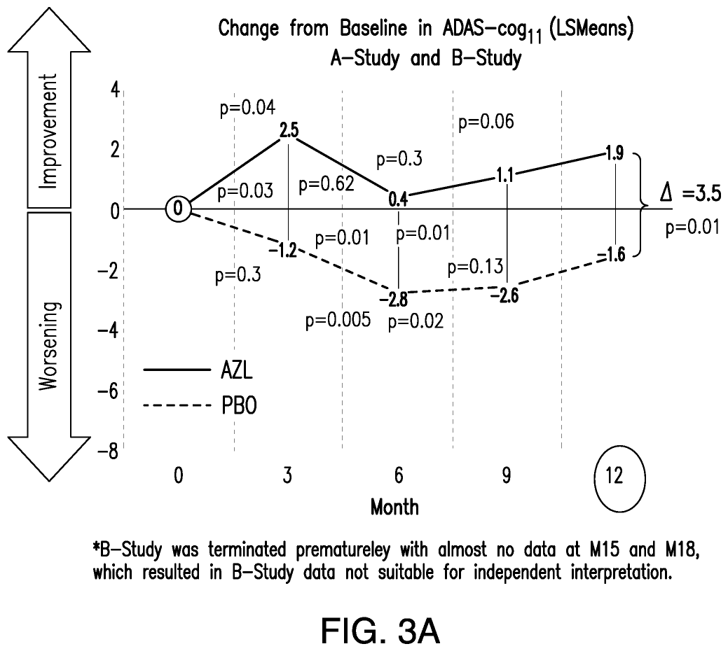

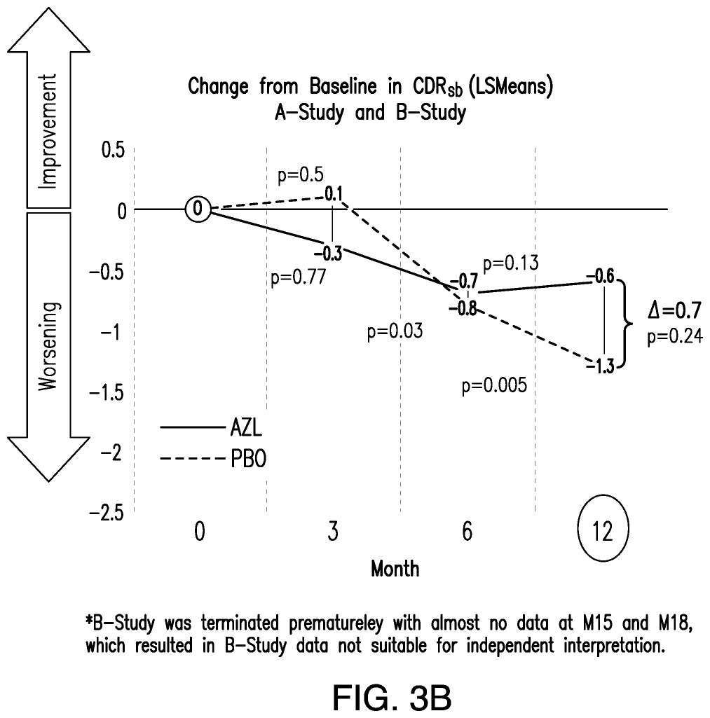

As shown in FIG. 3A, efficacy subgroup analysis of the A and B Studies revealed that patients with T2D (defined as patients with HbA1c of 6.5% or more at baseline) and AD treated with azeliragon (5 mg/day) did not show statistically significant cognitive decline (measured as change in ADAS-cog 11) when compared to baseline at any time during the study. In contrast, patients treated with placebo did show a statistically significant cognitive decline at month 6. Treatment differences between azeliragon-treated patients and placebo were significant (nominal p<0.05) at various time points, despite the small number of patients in this analysis.

FIG. 3B is 12-month graph displaying the change from baseline in CDR_{sb} for the subgroup of patients in A and B Studies having HbA1c of 6.5% or more at baseline.

As shown in FIG. 1, efficacy subgroup analysis of subjects in the A Study revealed that patients with T2D (defined as patients with HbA1c of 6.5% or more at baseline) and AD treated with azeliragon (5 mg/day) did not show statistically significant cognitive decline (measured as change in ADAS-cog 11) when compared to baseline at any time during the study. In contrast, patients treated with placebo did show a statistically significant cognitive decline relative to baseline at months 6, 9, 15 and 18. Starting at month 6 and continuing for months 9, 12, 15, and 18, treatment differences between azeliragon-treated patients and placebo were significant (nominal p<0.05), despite the small number of patients in this analysis.

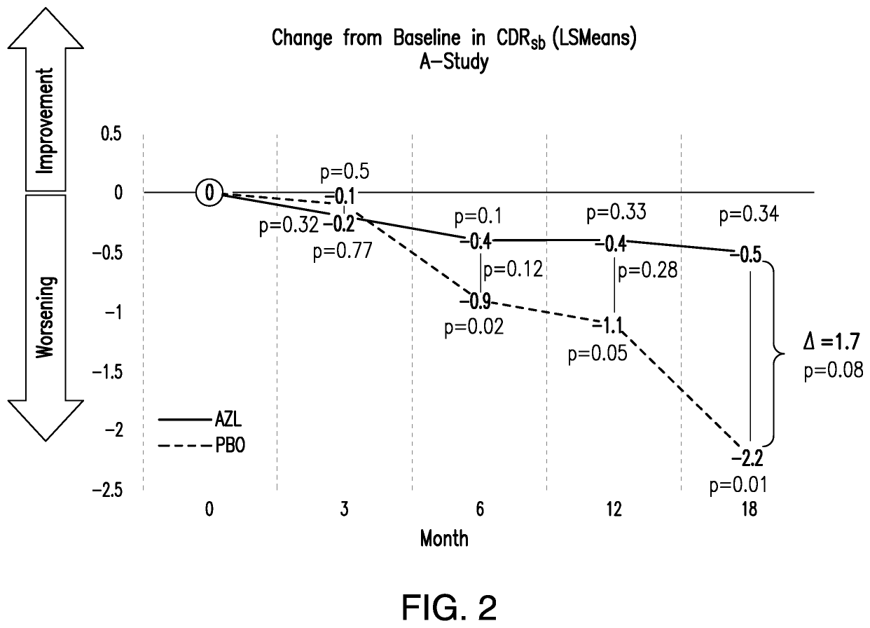

FIG. 2 is an 18-month graph displaying the change from baseline in CDR_{sb} for the subgroup of patients in the A Study having HbA1c of 6.5% or more at baseline. At 18 months, there was a pronounced separation in patients on active relative to patients on placebo, with patients on placebo appearing to decline at a faster rate.

The B Study was terminated early such that almost not data was collected for subjects at months 15 or 18. The number of subjects in the B Study having HbA1c of 6.5% or more at baseline who reached 12 months was so few that analysis of this data was not suitable for independent interpretation.

Results presented above were robust against the choice of statistical model, parametric or nonparametric analysis, or methodology for handling missing data. These results were confirmed by supportive analysis using the Wilcoxon methodology (valid for small samples and without making parametric assumptions), ANCOVA by visit, and ANCOVA with multiple-imputations for coping with missing data.

The improved cognition in treated subjects seemed to be independent of changes in glycemic control, pointing to potential changes in inflammation/vascular dysfunction.

The results of this analysis indicate a potential benefit of treatment with azeliragon for patients with poor glycemic control and cognitive impairment, but interpretation of the results is limited by the small number of subjects with both conditions participating in this study.

### Example 3

A further subgroup analysis of the data collected in the A Study produced the following results.

In contrast to the first subgroup's inclusion criteria, the inclusion criteria for this second subgroup was an HbA1c of 6.5% or more at any time during the study. Further, inclusion in this second subgroup analysis required stable diabetes therapy throughout the study, and insulin use was not allowed. Entry criteria for the entire A Study excluded patients having HbA1c>7.7%. For this second subgroup, Active N=26 and Placebo N=21.

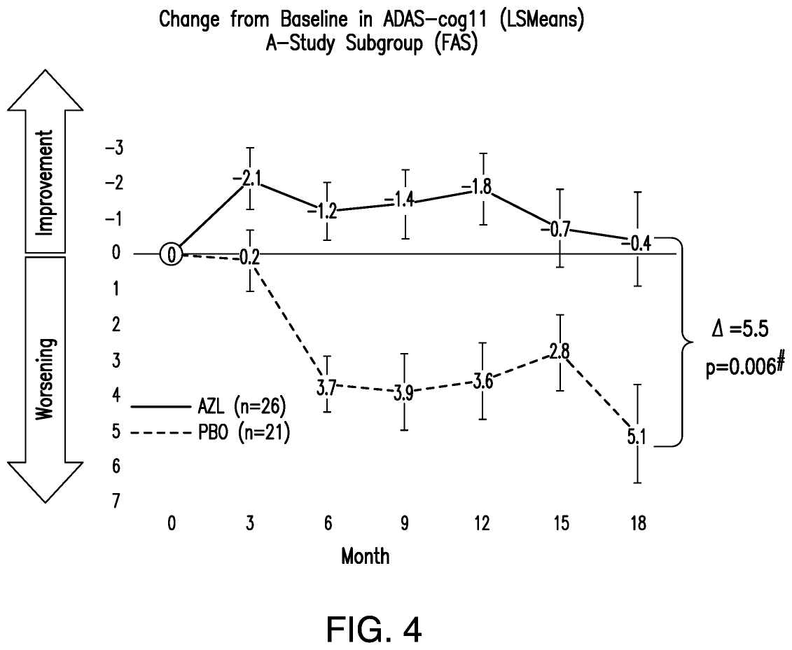

Analysis of this second subgroup produced similar results to those provided in FIGS. 1 and 2. The difference in active and placebo groups in ADAS-cog11 scores at month 18 for this second subgroup was 5.5 (nominal p=0.006) (LSMeans±SE) using the Full Analysis Set. See FIG. 4. The difference in active and placebo groups in CDRsb scores at month 18 for this second subgroup was 1.1 (nominal p=0.16) (LSMeans±SE) using the Full Analysis set.

Results in Table 1 and 2 below are Means±SE of the Full Analysis Set. Active N=26; Placebo N=21.

TABLE 1
Change from baseline in HbA1c (%) at month 18 for subjects
having HbA1c ≥ 6.5% at any time during the A Study

| | Active | Placebo |
|---|---|---|
| Change in HbA1c (%) | +0.04 (0.01) | +0.06 (0.015) |
| at Month 18 | | |

TABLE 2
Change from baseline in non-fasting glucose (mg/dL) at month 18
for subjects having HbA1c ≥ 6.5% at any time during the A Study

| | Active | Placebo |
|---|---|---|
| Change in Non-fasting Glucose | −7.0 (11) | +3.2 (8) |
| (mg/dL) at Month 18 | | |

The results in Tables 1 and 2 show that for the second subgroup cognitive improvement in ADAS-cog11 score for the active group was not accompanied by a significant reduction in HbA1c or non-fasting glucose after 18 months, thereby suggesting that cognitive improvement cannot be explained by a significant or pronounced improvement in glycemic control.

1-20. (canceled)

21. A method of treating cognitive impairment in a subject having an elevated level of one or more RAGE ligands or improving cognitive function in a subject having an elevated level of one or more RAGE ligands comprising: administering to the subject an amount of less than 20 mg per day of COMPOUND I or a pharmaceutically acceptable salt thereof, wherein the subject is a human.

22. (canceled)

23. The method of claim 21, wherein cognitive impairment may include one or more of the following symptoms: impairment of short-term or long-term memory function, learning, mental flexibility, attention, executive function, spatial working memory, numerical working memory, picture recognition, word recognition, inspection time, mood, vigor, anger, hostility, confusion, and total mood disturbance.

24. The method of claim 21, wherein the subject with cognitive impairment ranges from having mild cognitive impairment (MCI) to severe cognitive dysfunction.

25. The method of claim 24, wherein the subject with cognitive impairment may perform daily activities without assistance.

26. The method of claim 24, wherein the subject with cognitive impairment may suffer from dementia of Alzheimer's type, or mild or moderate Alzheimer's disease.

27. The method of claim 24, wherein the subject with cognitive impairment may satisfy one or more criteria (such as ADAS-cog, MMSE, CDR-sb, ADCS-iADL) for mild cognitive impairment (MCI), or for mild Alzheimer's disease, or for moderate Alzheimer's disease, or for vascular dementia.

28-30. (canceled)

31. The method of claim 21, wherein the subject has had at least one hypoglycemic event or severe hypoglycemic event in their lifetime.

32. (canceled)

33. The method of claim 21, wherein COMPOUND I or a pharmaceutically acceptable salt thereof is administered once a day, and the amount administered is 5 mg.

34. The method of claim 21, wherein the treatment results in a reduction in the rate of deterioration of one or more measures of cognitive function.

35-37. (canceled)

38. The method of claim 21, wherein the elevated RAGE ligand is selected from the group consisting of advanced glycation endproducts (AGEs), S100, calgranulin, EN-RAGE, β-amyloid (including Abeta (1-40)), CML (N^{ε}-Carboxymethyl lysine), high mobility group protein B1 (HMGB1), and amphoterin.

39. The method of claim 38, wherein an elevated level of a RAGE ligand is the upper quarter of a plasma range in humans or relative to a healthy control in humans.

40. The method of claim 38, wherein the elevated RAGE ligand is Abeta (1-40) and the plasma level of Abeta (1-40) in the subject is above 160 pg/mL.

41. A method of treating cognitive impairment or improving cognition in a subject having an elevated level of one or more inflammatory biomarkers comprising: administering to the subject an amount of less than 20 mg per day of COMPOUND I or a pharmaceutically acceptable salt thereof, wherein the level of one or more inflammatory biomarkers in the subject is reduced, and wherein the subject is a human.

42. (canceled)

43. The method of claim 41, wherein cognitive impairment may include one or more of the following symptoms: impairment of short-term or long-term memory function, learning, mental flexibility, attention, executive function, spatial working memory, numerical working memory, picture recognition, word recognition, inspection time, mood, vigor, anger, hostility, confusion, and total mood disturbance.

44. The method of claim 41, wherein the subject with cognitive impairment ranges from having mild cognitive impairment (MCI) to severe cognitive dysfunction.

45. The method of claim 44, wherein the subject with cognitive impairment may perform daily activities without assistance.

46. The method of claim 44, wherein the subject with cognitive impairment may suffer from dementia of Alzheimer's type, or mild or moderate Alzheimer's disease.

47. The method of claim 44, wherein the subject with cognitive impairment may satisfy one or more criteria (such as ADAS-cog, MMSE, CDR-sb, ADCS-iADL) for mild cognitive impairment (MCI), or for mild Alzheimer's disease, or for moderate Alzheimer's disease, or for vascular dementia.

48-50. (canceled)

51. The method of claim 41, wherein the subject has had at least one hypoglycemic event or severe hypoglycemic event in their lifetime.

52. (canceled)

53. The method of claim 41, wherein COMPOUND I or a pharmaceutically acceptable salt thereof is administered once a day, and the amount administered is 5 mg.

54. The method of claim 41, wherein the treatment results in a reduction in the rate of deterioration of one or more measures of cognitive function.

55-57. (canceled)

58. The method of claim 41, wherein the inflammatory biomarker is selected from the group consisting of IL2, IL6, IL12, IFNg, CD40L, MIP-1, and TNFb.

59. The method of claim 58, wherein the blood plasma level of one or more inflammatory biomarkers is reduced by at least 5%, 10%, 20%, 30%, 40%, 50%, 60%, 70%, or 80% relative to a baseline level.

60. The method of claim 58, wherein the blood plasma level of one or more of IL2, IL6, IL12, IFNg, and TNFb is reduced by at least 5%, 10%, 20%, 30%, 40%, 50%, 60%, 70%, or 80% relative to a baseline level.

61-89. (canceled)
